SEQUENCE LISTING (84 sequences)

SEQ ID NO: 1 - protein, 39 aa, Artificial Sequence
  Description of Artificial Sequence Amino Acid Sequence for the Generation of Antibodies
MSNITDPQMWDFDDLNFTGMPPADEDYSPCMLETETLNK

SEQ ID NO: 2 - protein, 48 aa, Artificial Sequence
  Description of Artificial Sequence Amino Acid Sequence for the Generation of Antibodies
MEDFNMESDSFEDFWKGEDLSNYSYSSTLPPFLLDAAPCEPESLEINK

SEQ ID NO: 3 - protein, 53 aa, Artificial Sequence
  Description of Artificial Sequence Amino Acid Sequence for the Generation of Antibodies
MVLEVSDHQVLNDAEVAALLENFSSSYDYGENESDSCCTSPPCPQDFSLNFDR

SEQ ID NO: 4 - protein, 39 aa, Artificial Sequence
  Description of Artificial Sequence Amino Acid Sequence for the Generation of Antibodies
MEGISIYTSDNYTEEMGSGDYDSMKEPCFREENANFNKI

SEQ ID NO: 5 - protein, 54 aa, Artificial Sequence
  Description of Artificial Sequence Amino Acid Sequence for the Generation of Antibodies
MNYPLTLEMDLENLEDLFWELDRLDNYNDTSLVENHLCPATGPLMASFKAVFVP

SEQ ID NO: 6 - protein, 32 aa, Artificial Sequence
  Description of Artificial Sequence Amino Acid Sequence for the Generation of Antibodies
MAEHDYHEDYGFSSFNDSSQEEHQDFLQFSKV

SEQ ID NO: 7 - protein, 34 aa, Artificial Sequence
  Description of Artificial Sequence Amino Acid Sequence for the Generation of Antibodies
METPNTTEDYDTTTEFDYGDATPCQKVNERAFGA

SEQ ID NO: 8 - protein, 42 aa, Artificial Sequence
  Description of Artificial Sequence Amino Acid Sequence for the Generation of Antibodies
MLSTSRSRFIRNTNESGEEVTTFFDYDYGAPCHKFDVKQIGA

SEQ ID NO: 9 - protein, 34 aa, Artificial Sequence
  Description of Artificial Sequence Amino Acid Sequence for the Generation of Antibodies
MTTSLDTVETFGTTSYYDDVGLLCEKADTRALMA

SEQ ID NO: 10 - protein, 39 aa, Artificial Sequence
  Description of Artificial Sequence Amino Acid Sequence for the Generation of Antibodies
MNPTDIADTTLDESIYSNYYLYESIPKPCTKEGIKAFGE

SEQ ID NO: 11 - protein, 30 aa, Artificial Sequence
  Description of Artificial Sequence Amino Acid Sequence for the Generation of Antibodies
MDYQVSSPIYDINYYTSEPCQKINVKQIAA

SEQ ID NO: 12 - protein, 47 aa, Artificial Sequence
  Description of Artificial Sequence Amino Acid Sequence for the Generation of Antibodies
MSGESMNFSDVFDSSEDYFVSVNTSYYSVDSEMLLCSLQEVRQFSRL

SEQ ID NO: 13 - protein, 42 aa, Artificial Sequence
  Description of Artificial Sequence Amino Acid Sequence for the Generation of Antibodies
MNFSDVFDSSEDYFVSVNTSYYSVDSEMLLCSLQEVRQFSRL

SEQ ID NO: 14 - protein, 59 aa, Artificial Sequence
  Description of Artificial Sequence Amino Acid Sequence for the Generation of Antibodies
MDLGKPMKSVLVVALLVIFQVCLCQDEVTDDYIGDNTTVDYTLFESLCSKKDVRNFKAW

SEQ ID NO: 15 - protein, 35 aa, Artificial Sequence
  Description of Artificial Sequence Amino Acid Sequence for the Generation of Antibodies
MDYTLDLSVTTVTDYYYPDIFSSPCDAELIQTNGK

SEQ ID NO: 16 - protein, 49 aa, Artificial Sequence
  Description of Artificial Sequence Amino Acid Sequence for the Generation of Antibodies
MTPTDFTSPIPNMADDYGSESTSSMEDYVNFNFTDFYCEKNNVRQFASH

SEQ ID NO: 17 - protein, 37 aa, Artificial Sequence
  Description of Artificial Sequence Amino Acid Sequence for the Generation of Antibodies
MADDYGSESTSSMEDYVNFNFTDFYCEKNNVRQFASH

SEQ ID NO: 18 - protein, 52 aa, Artificial Sequence
  Description of Artificial Sequence Amino Acid Sequence for the Generation of Antibodies
MGTEATEQVSWGHYSGDEEDAYSAEPLPELCYKADVQAFSRAFQPSVSLTVA

SEQ ID NO: 19 - protein, 42 aa, Artificial Sequence
  Description of Artificial Sequence Amino Acid Sequence for the Generation of Antibodies
MALEQNQSTDYYYEENEMNGTYDYSQYELICIKEDVREFAKV

SEQ ID NO: 20 - protein, 31 aa, Artificial Sequence
  Description of Artificial Sequence Amino Acid Sequence for the Generation of Antibodies
MESSGNPESTTFFYYDLQSQPCENQAWVFAT

SEQ ID NO: 21 - protein, 31 aa, Artificial Sequence
  Description of Artificial Sequence Amino Acid Sequence for the Generation of Antibodies
MDQFPESVTENFEYDDLAEACYIGDIVVFGT

SEQ ID NO: 22 - protein, 49 aa, Artificial Sequence
  Description of Artificial Sequence Amino Acid Sequence for the Generation of Antibodies
MAATASPQPLATEDADSENSSFYYYDYLDEVAFMLCRKDAVVSFGKVFL

SEQ ID NO: 23 - protein, 26 aa, Artificial Sequence
  Description of Artificial Sequence Amino Acid Sequence for the Generation of Antibodies
RQAYHPNNSSPVCYEVLGNDTAKWRM

SEQ ID NO: 24 - protein, 14 aa, Artificial Sequence [sequence omitted]

SEQ ID NO: 25 - protein, 25 aa, Artificial Sequence
  Description of Artificial Sequence Amino Acid Sequence for the Generation of Antibodies
RRTVYSSNVSPACYEDMGNNTANWR

SEQ ID NO: 26 - protein, 14 aa, Artificial Sequence
  Description of Artificial Sequence Amino Acid Sequence for the Generation of Antibodies
CFRRTVYSSNVSPA

SEQ ID NO: 27 - protein, 23 aa, Artificial Sequence
  Description of Artificial Sequence Amino Acid Sequence for the Generation of Antibodies
LSAHHDERLNATHCQYNFPQVGR

SEQ ID NO: 28 - protein, 14 aa, Artificial Sequence
  Description of Artificial Sequence Amino Acid Sequence for the Generation of Antibodies
CLSAHHDERLNATH

SEQ ID NO: 29 - protein, 25 aa, Artificial Sequence
  Description of Artificial Sequence Amino Acid Sequence for the Generation of Antibodies
NVSEADDRYICDRFYPNDLWVVVFQ

SEQ ID NO: 30 - protein, 16 aa, Artificial Sequence
  Description of Artificial Sequence Amino Acid Sequence for the Generation of Antibodies
DRFYPNDLWVVVFQFC

SEQ ID NO: 31 - protein, 31 aa, Artificial Sequence
  Description of Artificial Sequence Amino Acid Sequence for the Generation of Antibodies
KVSQGHHNNSLPRCTFSQENQAETHAWFTSR

SEQ ID NO: 32 - protein, 18 aa, Artificial Sequence
  Description of Artificial Sequence Amino Acid Sequence for the Generation of Antibodies
TFSQENQAETHAWFTSRC

SEQ ID NO: 33 - protein, 23 aa, Artificial Sequence
  Description of Artificial Sequence Amino Acid Sequence for the Generation of Antibodies
PQIIYGNVFNLDKLICGYHDEAI

SEQ ID NO: 34 - protein, 26 aa, Artificial Sequence
  Description of Artificial Sequence Amino Acid Sequence for the Generation of Antibodies
SKTQWEFTHHTCSLHFPHESLREWKL

SEQ ID NO: 35 - protein, 15 aa, Artificial Sequence
  Description of Artificial Sequence Amino Acid Sequence for the Generation of Antibodies
SLHFPHESLREWKLC

SEQ ID NO: 36 - protein, 28 aa, Artificial Sequence
  Description of Artificial Sequence Amino Acid Sequence for the Generation of Antibodies
TKCQKEDSVYVCGPYFPRGWNNFHTIMR

SEQ ID NO: 37 - protein, 18 aa, Artificial Sequence
  Description of Artificial Sequence Amino Acid Sequence for the Generation of Antibodies
GPYFPRGWNNFHTIMRNC

SEQ ID NO: 38 - protein, 32 aa, Artificial Sequence
  Description of Artificial Sequence Amino Acid Sequence for the Generation of Antibodies
YETEELFEETLCSALYPEDTVYSWRHFHTLRM

SEQ ID NO: 39 - protein, 22 aa, Artificial Sequence
  Description of Artificial Sequence Amino Acid Sequence for the Generation of Antibodies
SALYPEDTVYSWRHFHTLRMTC

SEQ ID NO: 40 - protein, 31 aa, Artificial Sequence
  Description of Artificial Sequence Amino Acid Sequence for the Generation of Antibodies
STCYTERNHTYCKTKYSLNSTTWKVLSSLEI

SEQ ID NO: 41 - protein, 21 aa, Artificial Sequence
  Description of Artificial Sequence Amino Acid Sequence for the Generation of Antibodies
KTKYSLNSTTWKVLSSLEINC

SEQ ID NO: 42 - protein, 32 aa, Artificial Sequence
  Description of Artificial Sequence Amino Acid Sequence for the Generation of Antibodies
TRSQKEGLHYTCSSHFPYSQYQFWKNFQTLKI

SEQ ID NO: 43 - protein, 22 aa, Artificial Sequence
  Description of Artificial Sequence Amino Acid Sequence for the Generation of Antibodies
SSHFPYSQYQFWKNFQTLKIVC

SEQ ID NO: 44 - protein, 30 aa, Artificial Sequence
  Description of Artificial Sequence Amino Acid Sequence for the Generation of Antibodies
STFVFNQKYNTQGSDVCEPKYQTVSEPIRW

SEQ ID NO: 45 - protein, 15 aa, Artificial Sequence
  Description of Artificial Sequence Amino Acid Sequence for the Generation of Antibodies
EPKYQTVSEPIRWKC

SEQ ID NO: 46 - protein, 28 aa, Artificial Sequence
  Description of Artificial Sequence Amino Acid Sequence for the Generation of Antibodies
PELLYSDLQRSSSEQAMRCSLITEHVEA

SEQ ID NO: 47 - protein, 19 aa, Artificial Sequence [sequence omitted]

SEQ ID NO: 48 - protein, 31 aa, Artificial Sequence
  Description of Artificial Sequence Amino Acid Sequence for the Generation of Antibodies
YQVASEDGVLQCYSFYNQQTLKWKIFTNFKM

SEQ ID NO: 49 - protein, 20 aa, Artificial Sequence
  Description of Artificial Sequence Amino Acid Sequence for the Generation of Antibodies
YSFYNQQTLKWKIFTNFKMC

SEQ ID NO: 50 - protein, 29 aa, Artificial Sequence
  Description of Artificial Sequence Amino Acid Sequence for the Generation of Antibodies
PEILYSQIKEESGIAICTMVYPSDESTKL

SEQ ID NO: 51 - protein, 14 aa, Artificial Sequence
  Description of Artificial Sequence Amino Acid Sequence for the Generation of Antibodies
CTMVYPSDESTKLK

SEQ ID NO: 52 - protein, 24 aa, Artificial Sequence
  Description of Artificial Sequence Amino Acid Sequence for the Generation of Antibodies
SQDGQREGQRRCRLIFPEGLTQTV

SEQ ID NO: 53 - protein, 13 aa, Artificial Sequence
  Description of Artificial Sequence Amino Acid Sequence for the Generation of Antibodies
FSQDGQREGQRRC

SEQ ID NO: 54 - protein, 22 aa, Artificial Sequence
  Description of Artificial Sequence Amino Acid Sequence for the Generation of Antibodies
TVNDNARCIPIFPRYLGTSMKA

SEQ ID NO: 55 - protein, 15 aa, Artificial Sequence
  Description of Artificial Sequence Amino Acid Sequence for the Generation of Antibodies
CIPIFPRYLGTSMKA

SEQ ID NO: 56 - protein, 23 aa, Artificial Sequence
  Description of Artificial Sequence Amino Acid Sequence for the Generation of Antibodies
HKVLSSGCDYSELTWYLTSVYQH

SEQ ID NO: 57 - protein, 16 aa, Artificial Sequence [sequence omitted]

SEQ ID NO: 58 - protein, 28 aa, Artificial Sequence
  Description of Artificial Sequence Amino Acid Sequence for the Generation of Antibodies
TKQKENECLGDYPEVLQEIWPVLRNVET

SEQ ID NO: 59 - protein, 20 aa, Artificial Sequence
  Description of Artificial Sequence Amino Acid Sequence for the Generation of Antibodies
LGDYPEVLQEIWPVLRNVET

SEQ ID NO: 60 - protein, 33 aa, Artificial Sequence
  Description of Artificial Sequence Amino Acid Sequence for the Generation of Antibodies
QTHENPKGVWNCHADFGGHGTIWKLFLRFQQNL

SEQ ID NO: 61 - protein, 21 aa, Artificial Sequence
  Description of Artificial Sequence Amino Acid Sequence for the Generation of Antibodies
HADFGGHGTIWKLFLRFQQNC

SEQ ID NO: 62 - protein, 355 aa, Artificial Sequence
  Description of Artificial Sequence Amino Acid Sequence for the Generation of Antibodies
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 63 - protein, 374 aa, Artificial Sequence
  Description of Artificial Sequence Amino Acid Sequence for the Generation of Antibodies
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 64 - protein, 360 aa, Artificial Sequence
  Description of Artificial Sequence Amino Acid Sequence for the Generation of Antibodies
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 65 - protein, 355 aa, Artificial Sequence
  Description of Artificial Sequence Amino Acid Sequence for the Generation of Antibodies
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 66 - protein, 360 aa, Artificial Sequence
  Description of Artificial Sequence Amino Acid Sequence for the Generation of Antibodies
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 67 - protein, 352 aa, Artificial Sequence [sequence omitted]

SEQ ID NO: 68 - protein, 374 aa, Artificial Sequence
  Description of Artificial Sequence Amino Acid Sequence for the Generation of Antibodies
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 69 - protein, 369 aa, Artificial Sequence
  Description of Artificial Sequence Amino Acid Sequence for the Generation of Antibodies
MNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 70 - protein, 378 aa, Artificial Sequence
  Description of Artificial Sequence Amino Acid Sequence for the Generation of Antibodies
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 71 - protein, 355 aa, Artificial Sequence
  Description of Artificial Sequence Amino Acid Sequence for the Generation of Antibodies
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 72 - protein, 369 aa, Artificial Sequence
  Description of Artificial Sequence Amino Acid Sequence for the Generation of Antibodies
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 73 - protein, 357 aa, Artificial Sequence
  Description of Artificial Sequence Amino Acid Sequence for the Generation of Antibodies
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 74 - protein, 362 aa, Artificial Sequence
  Description of Artificial Sequence Amino Acid Sequence for the Generation of Antibodies
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSSPSGPQPRRGCPRRPRLSSCSAPTETHSLSWDN

SEQ ID NO: 75 - protein, 350 aa, Artificial Sequence
  Description of Artificial Sequence Amino Acid Sequence for the Generation of Antibodies
MALEQNQSTDYYYEENEMNGTYDYSQYELICIKEDVREFAKVFLPVFLTIVFVIGLAGNSMVVAIYAYYKKQRTKTDVYILNLAVADLLLLFTLPFWAVNAVHGWVLGKIMCKITSALYTLNFVSGMQFLACISIDRYVAVTKVPSQSGVGKPCWIICFCVWMAAILLSIPQLVFYTVNDNARCIPIFPRYLGTSMKALIQMLEICIGFVVPFLIMGVCYFITARTLMKMPNIKISRPLKVLLTVVIVFIVTQLPYNIVKFCRAIDIIYSLITSCNMSKRMDIAIQVTESIALFHSCLNPILYVFMGASFKNYVMKVAKKYGSWRRQRQSVEEFPFDSEGPTEPTSTFSI

SEQ ID NO: 76 - protein, 350 aa, Artificial Sequence [sequence omitted]

SEQ ID NO: 77 - protein, 360 aa, Artificial Sequence
  Description of Artificial Sequence Amino Acid Sequence for the Generation of Antibodies
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 78 - protein, 368 aa, Artificial Sequence
  Description of Artificial Sequence Amino Acid Sequence for the Generation of Antibodies
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 79 - protein, 352 aa, Artificial Sequence
  Description of Artificial Sequence Amino Acid Sequence for the Generation of Antibodies
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 80 - protein, 372 aa, Artificial Sequence
  Description of Artificial Sequence Amino Acid Sequence for the Generation of Antibodies
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 81 - protein, 342 aa, Artificial Sequence
  Description of Artificial Sequence Amino Acid Sequence for the Generation of Antibodies
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 82 - protein, 355 aa, Artificial Sequence
  Description of Artificial Sequence Amino Acid Sequence for the Generation of Antibodies
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 83 - protein, 333 aa, Artificial Sequence
  Description of Artificial Sequence Amino Acid Sequence for the Generation of Antibodies
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 84 - protein, 384 aa, Artificial Sequence
  Description of Artificial Sequence Amino Acid Sequence for the Generation of Antibodies
MAATASPQPLATEDADSENSSFYYYDYLDEVAFMLCRKDAVVSFGKVFLPVFYSLIFVLGLSGNLLLLMVLLRYVPRRRMVEIYLLNLAISNLLFLVTLPFWGISVAWHWVFGSFLCKMVSTLYTINFYSGIFFISCMSLDKYLEIVHAQPYHRLRTRAKSLLLATIVWAVSLAVSIPDMVFVQTHENPKGVWNCHADFGGHGTIWKLFLRFQQNLLGFLLPLLAMIFFYSRIGCVLVRLRPAGQGRALKIAAALVVAFFVLWFPYNLTLFLHTLLDLQVFGNCEVSQHLDYALQVTESIAFLHCCFSPILYAFSSHRFRQYLKAFLAAVLGWHLAPGTAQASLSSCSESSILTAQEEMTGMNDLGERQSENYPNKEDVGNKSA

(19) United States
(12) Patent Application Publication
(10) Pub. No.: US 2003/0186889 A1
(43) Pub. Date: Oct. 2, 2003

(54) Diagnostic and medicament for analysing the cell surface proteome of tumour and inflammatory cells and for treating tumorous and inflammatory diseases, preferably using a specific chemokine receptor analysis and the chemokine receptor-ligand interaction

(76) Inventors: Wolf-Georg Forssmann, Hannover (DE); Ulf Forssmann, Wunstorf (DE); Knut Adermann, Hannover (DE); Aleksandra Heitland, Hannover (DE); Nicolaj Spodsberg, Hannover (DE)

Correspondence Address: JACOBSON HOLMAN PLLC, 400 SEVENTH STREET N.W., SUITE 600, WASHINGTON, DC, 20004, US

(21) Appl. No.: 10/239,423

(22) PCT Filed: Apr. 2, 2001
(86) PCT No.: PCT/EP01/03708

(30) Foreign Application Priority Data
Mar. 31, 2000 (DE) 10016013.1

(51) Int. Cl. G01N 33/574; A61K 39/395; A61K 38/10

(52) U.S. Cl. 514/14; 435/7.23; 424/143.1

(57) ABSTRACT

The present invention relates to the provision of a medicament and of a diagnostic agent obtained by proteome analysis, preferably containing at least two different chemokine receptor ligands or chemokine receptor antibodies, and further to the use of at least two different chemokine receptor ligands, chemokine receptor antibodies and/or two different chemokine receptors. As inhibitors, the medicament preferably contains ligands or antibodies of at least two chemokine receptors or the related algorithms of the surface chemokine receptor proteome, and the use of at least one chemokine receptor ligand and/or chemokine receptor, peptides and antibodies and their use for the diagnostics and therapy of tumor diseases and inflammatory diseases. By way of analogy, clusters of analyzed tumor cell surface proteomes, such as ectoproteases, adhesion molecules or various receptor types, can also be used.

The invention relates to a method for the purpose of diagnosis and therapy and the medical and industrial use of chemokines and their corresponding receptors, their antagonists including antibodies was found to inhibit cancer growth including metastatic spread, and to suppress inflammatory and auto-immune diseases. The method is based on the finding that chemokines act on specific tumor and inflammation cells via autocrine, paracrine and endocrine routes through the disease-specific constellation of the chemokine receptor proteome. Primary and secondary tumors as well as specific inflammation cells are controlled with respect to their migration and proliferation behavior. By diagnostically detecting the chemokines whose expression and regulation is locally increased and the presence of the chemokine receptor compositions, the possibility arises of critically suppressing or completely preventing cancer growth, metastatic spread of tumors as well as inflammatory and auto-immune diseases.

The present invention relates to the provision of a medicament and of a diagnostic agent obtained by proteome analysis, preferably containing at least two different chemokine receptor ligands or chemokine receptor antibodies, and further to the use of at least two different chemokine receptor ligands, chemokine receptor antibodies and/or two different chemokine receptors. The medicament comprises the use of inhibitors, ligands or antibodies of at least two chemokine receptors or the related algorithms of the surface chemokine receptor proteome, and the use of at least one chemokine receptor ligand and/or chemokine receptor, peptides and antibodies and their use for the diagnostics and therapy of tumor diseases and inflammatory diseases. By way of analogy, clusters of analyzed tumor cell surface proteomes, such as ectoproteases, adhesion molecules or various receptor types, can also be used.

In recent time, it has been found more and more often that substances of the regulation of cellular growth and cell migration are of great diagnostic and therapeutic interest, and that a large number of unknown factors are likely to exist which control cancer growth, while primary and secondary tumors and the invaded organs are interacting. Surprisingly, these factors are of clinically useful importance. They include, in particular, the CC and CXC type chemokines, preferably, for example, HCC-1 to HCC-3 and SDF-1 (Schulz-Knappe P. et al., J. Exp. Med. 183: 295, 1996; Pardigol A. et al., Proc. Natl. Acad. Sci. USA 95: 6308, 1998; Nagasawa T. et al., Proc. Natl. Acad. Sci. USA 91: 2305, 1994). Thus, in recent works, it could be demonstrated that cell lines of tumor cells, inter alia, possess the receptors for the known chemokines MCP-1 and RANTES (Wang J. M. et al., Int. J. Cancer 75: 900, 1998). Thus, these tumor cell lines can respond to specific chemokines through stimulation of the receptors. When the corresponding chemokine peptide is formed in appropriate cells, tissues and organs, an autocrine, paracrine or endocrine reaction may occur, influencing cell migration and cell proliferation. Thus, kidney metastases are said to be formed preferentially from those tumors which possess receptors for MCP-1 and RANTES because a strong expression of these chemokines is observed in the kidney (Wang J. M. et al., Int. J. Cancer 75: 900, 1998). In further recent works, it was also established that certain chemokines inhibit tumor growth (Wang J. M. et al., J. Interferon Cytokine Res. 16: 53, 1996), so that tumor growth may also be influenced negatively by a chemokine administration. The role of chemokines in the migration of tumor cells was observed in breast carcinoma cell lines (Youngs et al., Int. J. Cancer 71: 257, 1997). The control of angiogenesis is also influenced positively or negatively by various cytokines. Thus, the chemokine receptor CXCR4, through which the chemokine SDF-1 displays its activity, is essential to the vascularization of the gastrointestinal tract (Tachibana K. et al., Nature 393: 591, 1998). Thus, these factors may also be of critical importance within the scope of the paracrine control of the vascularization of tumors and thus to tumor maintenance.

The results known to date with respect to the chemokine system and tumors are always based on the observation of individual chemokine receptors. However, because over 15 chemokine receptors have become known in the meantime with over 40 related chemokines, the human chemokine system is extremely complex and moreover redundant. Thus, the previously applied diagnostic and therapeutic method approaches are not optimal for performing specific diagnostics and a specific therapy based on the chemokine system. With the method applied according to the invention, surprisingly, a solution to these problems can be found, thus for the first time allowing reliable diagnostics and therapy of tumor and inflammatory diseases based on the chemokine system or other tumor surface proteome clusters.

One object of the invention is to improve the diagnostics of tumors and of the presence of inflammatory processes. Another object is to provide an improved treatment of tumors and inflammatory diseases.

This object is achieved by a diagnostic agent and a medicament according to the invention.

The diagnostic agent according to the invention contains at least two different ligands of receptors which are involved in a pathological process.

Preferably, the diagnostic agent according to the invention contains at least two different chemokine receptor ligands, such as protein peptide structures, which interact with chemokine receptors (or other tumor cell surface proteins), namely chemokine receptor ligands, chemokine receptor antagonists and/or chemokine receptor antibodies. Preferably, the chemokine receptor ligands are chemokines, chemokine derivatives, agonists or antagonists of chemokine receptors, antibodies or antibody fragments which at least partially block the binding site of the chemokine receptor and surprisingly result in an inhibition of tumor growth.

More preferably, the chemokines are selected from the group consisting of the natural chemokines C, CC, CXC, CX₃C, their analogues, binding proteins and antibodies which bind to the specific receptors in accordance with the chemokines mentioned.

According to the invention, the chemokine receptors are detected and analyzed as a whole or partial proteome including the corresponding chemokine receptor ligands in primary and secondary tumors and circulating single cells, preferably by (1) immunochemical methods (immunohistochemistry using serial sections or multiple successive or simultaneous single sections, FACS analysis), and (2) additionally or alternatively the expression on the transcriptional level by molecular-biological methods (PCR or Northern analysis, in-situ hybridization). Clusters of analyzed tumor cell surface proteomes, such as ectoproteases, adhesion molecules or various receptor types, can also be employed according to the invention.

Closely related to the diagnostic agent according to the invention is the method according to the invention for recognizing receptors involved in pathological processes, wherein expression profiles are examined on the proteome level using cell-biological or cytochemical methods, especially by immunochemical methods, immunohistochemistry using serial sections or multiple successive or simultaneous single sections, FACS analysis and/or the expression of receptors on the transcriptional level by molecular-biological methods, especially PCR, Northern analysis and/or in-situ hybridization methods.

Thus, the present invention also relates to the use of at least two different chemokine receptor ligands and/or two different chemokine receptors for the diagnostic characterization of tumors, which is different for each tumor type and each individual tumor. In particular, the method according to the invention can be applied to tumors from the group consisting of colorectal tumors and prostatic tumors. However, tumors of other organ systems may also be approached diagnostically and thus therapeutically as well with the method according to the invention. In addition, said at least two different chemokine receptors and/or two different chemokine receptor ligands may also be employed for the diagnosis of inflammatory processes, such as organ rejection responses, and for the diagnosis of auto-immune diseases. Thus, especially tumors, inflammatory diseases and auto-immune diseases of the blood system, the lymph system, the cardiovascular system, the nervous system, the respiratory tract, the digestive tract, the endocrine system, the skin including integumentary appendages, the locomotor system and the urogenital tract including the kidney can be diagnosed.

In contrast to commonly known diagnostic methods, the diagnostic agent according to the invention enables an extension of the cytological characterization of tumor tissues, which may also be used to develop a specific therapy after the diagnosis. Of the chemokine receptors found (and by analogy of other tumor cell proteome clusters), the antagonists/agonists of the related chemokines and the specific chemokine receptor antibodies are employed for influencing the cellular growth. When doing so, an accelerated occurrence of tumor cell death (apoptosis) is surprisingly observed.

The present invention also relates to a medicament containing at least one inhibitor of at least two chemokine receptors.

The medicament according to the invention preferably contains antagonists of chemokine receptors, antibodies or antibody fragments which at least partially block the binding site of the chemokine receptor. However, a chemokine receptor/ligand interaction, preferably a protein/protein (peptide) interaction with non-specific molecules obtained from natural extracts, from synthetic or recombinantly prepared binding proteins and from other peptide-protein libraries, is also sufficient to bring about the surprising effect of the apoptosis of tumor cells.

The inhibitors of at least two chemokine receptors which may be used in the medicament according to the invention may be used for the preparation of a medicament for treating tumors, inflammatory diseases, auto-immune diseases of the bone marrow and other organs, graft rejection reactions. Thus, in particular, tumors, inflammatory diseases and auto-immune diseases of the blood system, the lymph system, the cardiovascular system, the nervous system, the respiratory tract, the digestive tract, the endocrine system, the skin including integumentary appendages, the locomotor system and the urogenital tract including the kidney can be treated.

Preferably, inhibitors or protein ligands are used which are selected from the group consisting of antagonists of chemokine receptors, antibodies or antibody fragments which at least partially block the binding site of the chemokine receptor.

The tumors which can be treated are especially selected from the group consisting of colorectal tumors, prostatic tumors and other tumor diseases of the blood system, lymph system, cardiovascular system, nervous system, respiratory tract, digestive tract, endocrine system, skin including integumentary appendages, locomotor system and urogenital tract including the kidney.

The inflammatory processes to be treated are especially selected from the group consisting of asthma bronchiale, chronic inflammatory bowel diseases, organ rejection and further inflammatory processes of the blood system, lymph system, cardiovascular system, nervous system, respiratory tract, digestive tract, endocrine system, skin including integumentary appendages, locomotor system and urogenital tract including the kidney.

The auto-immune diseases to be treated are especially selected from the group consisting of rheumatoid arthritis, lupus erythematodes and other chronic diseases of the blood system, lymph system, cardiovascular system, nervous system, respiratory tract, digestive tract, endocrine system, skin including integumentary appendages, locomotor system and urogenital tract including the kidney.

In the diagnosis of organ rejection reactions following organ transplantations, especially performed on leucocytes obtained from the circulation of patients, the use of only at least one chemokine receptor ligand and/or one chemokine receptor is also possible.

The invention further relates to the use of an inhibitor of a chemokine receptor for preparing a medicament for preventing or alleviating organ rejection reactions following organ transplantations, especially following transplantations of the heart, liver, kidney and pancreas as well as other organs, tissues and cell systems of the gastrointestinal tract, respiratory tract, urogenital tract, cardiovascular system, neuro-endocrine system, and the locomotor system as well as the blood and immune systems.

In addition to establishing new causalities in a pathological process, the method according to the invention can also be employed for the purpose of diagnosis and therapy. By means of the method according to the invention, for example, the use of chemokines and their corresponding receptors, their antagonists including antibodies was found to inhibit cancer growth including metastatic spread, and to suppress inflammatory and auto-immune diseases. The method according to the invention is based on the finding that chemokines act on specific tumor and inflammation cells via autocrine, paracrine and endocrine routes through the disease-specific constellation of the chemokine receptor proteome. Primary and secondary tumors as well as specific inflammation cells are controlled with respect to their migration and proliferation behavior. By diagnostically detecting the chemokines whose expression and regulation is locally increased and the presence of the chemokine receptor compositions, the possibility arises of critically suppressing or completely preventing cancer growth, metastatic spread of tumors as well as inflammatory and auto-immune diseases.

The peptides according to the invention having the SEQ ID NOS. 1 to 40 can be employed as epitopes for generating antibodies. These sequences according to the invention are (ID 1-40):

Amino acid sequences of the N-terminal domains of human chemokine receptors employed for the preparation of specific antibodies

1 CXCR1 X-MSNITDPQMWDFDDLNFTGMPPADEDYSPCMLETETLNK-Y

2 CXCR2 X-MEDFNMESDSFEDFWKGEDLSNYSYSSTLPPFLLDAAPCEPESLEINK-Y

3 CXCR3 X-MVLEVSDHQVLNDAEVAALLENFSSSYDYGENESDSCCTSPPCPQDFSLNFDR-Y

4 CXCR4 X-MEGISIYTSDNYTEEMGSGDYDSMKEPCFREENANFNKI-Y

5 CXCR5 X-MNYPLTLEMDLENLEDLFWELDRLDNYNDTSLVENHLCPATGPLMASFKAVFVP-Y

4 CXCR6 X-MAEHDYHEDYGFSSFNDSSQEEHQDFLQFSKV-Y

5 CCR1 X-METPNTTEDYDTTTEFDYGDATPCQKVNERAFGA-Y

6 CCR2 X-MLSTSRSRFIRNTNESGEEVTTFFDYDYGAPCHKFDVKQIGA-Y

7 CCR3 X-MTTSLDTVETFGTTSYYDDVGLLCEKADTRALMA-Y

8 CCR4 X-MNPTDIADTTLDESIYSNYYLYESIPKPCTKEGIKAFGE-Y

9 CCR5 X-MDYQVSSPIYDINYYTSEPCQKINVKQIAA-Y

10 CCR6a X-MSGESMNFSDVFDSSEDYFVSVNTSYYSVDSEMLLCSLQEVRQFSRL-Y

11 CCR6b X-MNFSDVFDSSEDYFVSVNTSYYSVDSEMLLCSLQEVRQFSRL-Y

12 CCR7 X-MDLGKPMKSVLVVALLVIFQVCLCQDEVTDDYIGDNTTVDYTLFESLCSKKDVRNFKAW-Y

13 CCR8 X-MDYTLDLSVTTVTDYYYPDIFSSPCDAELIQTNGK-Y

14 CCR9a X-MTPTDFTSPIPNMADDYGSESTSSMEDYVNFNFTDFYCEKNNVRQFASH-Y

15 CCR9b X-MADDYGSESTSSMEDYVNFNFTDFYCEKNNVRQFASH-Y

16 CCR10 X-MGTEATEQVSWGHYSGDEEDAYSAEPLPELCYKADVQAFSRAFQPSVSLTVA-Y

17 CCR11 X-MALEQNQSTDYYYEENEMNGTYDYSQYELICIKEDVREFAKV-Y

18 XCR1 X-MESSGNPESTTFFYYDLQSQPCENQAWVFAT-Y

19 CX3CR1 X-MDQFPESVTENFEYDDLAEACYIGDIVVFGT-Y

20 D6 X-MAATASPQPLATEDADSENSSFYYYDYLDEVAFMLCRKDAVVSFGKVFL-Y

Amino acid sequences of the 2nd extracellular loop domains of human chemokine receptors employed for the preparation of specific antibodies

21 CXCR1 X-RQAYHPNNSSPVCYEVLGNDTAKWRM-Y, especially CFRQAYHPNNSSPV

22 CXCR2 X-RRTVYSSNVSPACYEDMGNNTANWR-Y, especially CFRRTVYSSNVSPA

23 CXCR3 X-LSAHHDERLNATHCQYNFPQVGR-Y, especially CLSAHHDERLNATH

24 CXCR4 X-NVSEADDRYICDRFYPNDLWVVVFQ-Y, especially DRFYPNDLWVVVFQFC

25 CXCR5 X-KVSQGHHNNSLPRCTFSQENQAETHAWFTSR-Y, especially TFSQENQAETHAWFTSRC

26 CXCR6 X-PQIIYGNVFNLDKLICGYHDEAI-Y, especially

27 CCR1 X-SKTQWEFTHHTCSLHFPHESLREWKL-Y, especially SLHFPHESLREWKLC

28 CCR2 X-TKCQKEDSVYVCGPYFPRGWNNFHTIMR-Y, especially GPYFPRGWNNFHTIMRNC

29 CCR3 X-YETEELFEETLCSALYPEDTVYSWRHFHTLRM-Y, especially SALYPEDTVYSWRHFHTLRMTC

30 CCR4 X-STCYTERNHTYCKTKYSLNSTTWKVLSSLEI-Y, especially KTKYSLNSTTWKVLSSLEINC

31 CCR5 X-TRSQKEGLHYTCSSHFPYSQYQFWKNFQTLKI-Y, especially SSHFPYSQYQFWKNFQTLKIVC

32 CCR6 X-STFVFNQKYNTQGSDVCEPKYQTVSEPIRW-Y, especially EPKYQTVSEPIRWKC

33 CCR7 X-PELLYSDLQRSSSEQAMRCSLITEHVEA-Y, especially CPELLYSDLQRSSSEQAMR

34 CCR8 X-YQVASEDGVLQCYSFYNQQTLKWKIFTNFKM-Y, especially YSFYNQQTLKWKIFTNFKMC

35 CCR9 X-PEILYSQIKEESGIAICTMVYPSDESTKL-Y, especially CTMVYPSDESTKLK

36 CCR10 X-SQDGQREGQRRCRLIFPEGLTQTV-Y, especially FSQDGQREGQRRC

37 CCR11 X-TVNDNARCIPIFPRYLGTSMKA-Y, especially CIPIFPRYLGTSMKA

38 XCR1 X-HKVLSSGCDYSELTWYLTSVYQH-Y, especially DYSELTWYLTSVYQHC

39 CX3CR1 X-TKQKENECLGDYPEVLQEIWPVLRNVET-Y, especially LGDYPEVLQEIWPVLRNVET

40 D6 X-QTHENPKGVWNCHADFGGHGTIWKLFLRFQQNL-Y, especially HADFGGHGTIWKLFLRFQQNC

wherein X means an amino acid residue or a peptide residue of up to 30 amino acids, wherein Y means an amino acid residue or a peptide residue of up to 30 amino acids.

To prepare the antibodies, the peptides according to the invention (sequences according to ID 1-40), which are not normally immunogenic, are coupled to the protein carrier KLH (keyhole limpet hemocyanin). This coupling is effected by means of MBS (m-maleimidobenzoyl-N-hydroxysuccinimide ester) through a cysteine terminally integrated into the peptide, or directly by means of carbodiimide.

The antibodies are obtained with conventional methods by immunization, preferably of mice, rabbits etc. The methods for the preparation of monoclonal antibodies by molecular-biological methods, such as recombinant preparation, can also be used. The antibodies are purified by the known methods and galenically formulated for use.

Further, cell preparations, cell extracts and, in particular, membrane isolates from overexpressing, artificially transfected chemokine receptor bearing cells were used for generating such specific antibodies.

According to the invention, this included the use of proteins based on known sequences as epitopes for generating antibodies. These sequences are known (ID 41-63):

41 CCR1 ACCESSION P32246 355aa

1 METPNTTEDY DTTTEFDYGD ATPCQKVNER AFGAQLLPPL YSLVFVIGLV GNILVVLVLV

61 QYKRLKNMTS IYLLNLAISD LLFLFTLPFW IDYKLKDDWV FGDAMCKILS GFYYTGLYSE

121 IFFIILLTID RYLAIVHAVF ALRARTVTFG VITSIIIWAL AILASMPGLY FSKTQWEFTH

181 HTCSLHFPHE SLREWKLFQA LKLNLFGLVL PLLVMIICYT GIIKILLRRP NEKKSKAVRL

241 IFVIMIIFFL FWTPYNLTIL ISVFQDFLFT HECEQSRHLD LAVQVTEVIA YTHCCVNPVI

301 YAFVGERFRK YLRQLFHRRV AVHLVKWLPF LSVDRLERVS STSPSTGEHE LSAGF

42 CCR2a ACCESSION P41597 374aa

1 MLSTSRSRFI RNTNESGEEV TTFFDYDYGA PCHKFDVKQI GAQLLPPLYS LVFIFGFVGN

61 MLVVLILINC KKLKCLTDIY LLNLAISDLL FLITLPLWAH SAANEWVFGN AMCKLFTGLY

121 HIGYFGGIFF IILLTIDRYL AIVHAVFALK ARTVTFGVVT SVITWLVAVF ASVPGIIFTK

181 CQKEDSVYVC GPYFPRGWNN FHTIMRNILG LVLPLLIMVI CYSGILKTLL RCRNEKKRHR

241 AVRVIFTIMI VYFLFWTPYN IVILLNTFQE FFGLSNCEST SQLDQATQVT ETLGMTHCCI

301 NPIIYAFVGE KFRSLFHIAL GCRIAPLQKP VCGGPGVRPG KNVKVTTQGL LDGRGKGKSI

361 GRAPEASLQD KEGA

43 CCR2b ACCESSION NP₁₃000639 360aa

1 MLSTSRSRFI RNTNESGEEV TTFFDYDYGA PCHKFDVKQI GAQLLPPLYS LVFIFGFVGN

61 MLVVLILINC KKLKCLTDIY LLNLAISDLL FLITLPLWAH SAANEWVFGN AMCKLFTGLY

121 HIGYFGGIFF IILLTIDRYL AIVHAVFALK ARTVTFGVVT SVITWLVAVF ASVPGIIFTK

181 CQKEDSVYVC GPYFPRGWNN FHTIMRNILG LVLPLLIMVI CYSGILKTLL RCRNEKKRHR

241 AVRVIFTIMI VYFLFWTPYN IVILLNTFQE FFGLSNCEST SQLDQATQVT ETLGMTHCCI

301 NPIIYAFVGE KFRRYLSVFF RKHITKRFCK QCPVFYRETV DGVTSTNTPS TGEQEVSAGL

44 CCR3 ACCESSION P51677 355aa

1 MTTSLDTVET FGTTSYYDDV GLLCEKADTR ALMAQFVPPL YSLVFTVGLL GNVVVVMILI

61 KYRRLRIMTN IYLLNLAISD LLFLVTLPFW IHYVRGHNWV FGHGMCKLLS GFYHTGLYSE

121 IFFIILLTID RYLAIVHAVF ALRARTVTFG VITSIVTWGL AVLAALPEFI FYETEELFEE

181 TLCSALYPED TVYSWRHFHT LRMTIFCLVL PLLVMAICYT GIIKTLLRCP SKKKYKAIRL

241 IFVIMAVFFI FWTPYNVAIL LSSYQSILFG NDCERSKHLD LVMLVTEVIA YSHCCMNPVI

301 YAFVGERFRK YLRHFFHRHL LMHLGRYIPF LPSEKLERTS SVSPSTAEPE LSIVF

45 CCR4 ACCESSION P51679 360aa

1 MNPTDIADTT LDESIYSNYY LYESIPKPCT KEGIKAFGEL FLPPLYSLVF VFGLLGNSVV

61 VLVLFKYKRL RSMTDVYLLN LAISDLLFVF SLPFWGYYAA DQWVFGLGLC KMISWMYLVG

121 FYSGIFFVML MSIDRYLAIV HAVFSLRART LTYGVITSLA TWSVAVFASL PGFLFSTCYT

181 ERNHTYCKTK YSLNSTTWKV LSSLEINILG LVIPLGIMLF CYSMIIRTLQ HCKNEKKNKA

241 VKMIFAVVVL FLGFWTPYNI VLFLETLVEL EVLQDCTFER YLDYAIQATE TLAFVHCCLN

301 PIIYFFLGEK FRKYILQLFK TCRGLFVLCQ YCGLLQIYSA DTPSSSYTQS TMDHDLHDAL

361

46 CCR5 ACCESSION P51681 352aa

1 MDYQVSSPIY DINYYTSEPC QKINVKQIAA RLLPPLYSLV FIFGFVGNML VILILINCKR

61 LKSMTDIYLL NLAISDLFFL LTVPFWAHYA AAQWDFGNTM CQLLTGLYFI GFFSGIFFII

121 LLTIDRYLAV VHAVFALKAR TVTFGVVTSV ITWVVAVFAS LPGIIFTRSQ KEGLHYTCSS

181 HFPYSQYQFW KNFQTLKIVI LGLVLPLLVM VICYSGILKT LLRCRNEKKR HRAVRLIFTI

241 MIVYFLFWAP YNIVLLLNTF QEFFGLNNCS SSNRLDQAMQ VTETLGMTHC CINPIIYAFV

301 GEKFRNYLLV FFQKHIAKRF CKCCSIFQQE APERASSVYT RSTGEQEISV GL

47 CCR6a ACCESSION P51684 374aa

1 MSGESMNFSD VFDSSEDYFV SVNTSYYSVD SEMLLCSLQE VRQFSRLFVP IAYSLICVFG

61 LLGNILVVIT FAFYKKARSM TDVYLLNMAI ADILFVLTLP FWAVSHATGA WVFSNATCKL

121 LKGIYAINFN CGMLLLTCIS MDRYIAIVQA TKSFRLRSRT LPRSKIICLV VWGLSVIISS

181 STFVFNQKYN TQGSDVCEPK YQTVSEPIRW KLLMLGLELL FGFFIPLMFM IFCYTFIVKT

241 LVQAQNSKRH KAIRVIIAVV LVFLACQIPH NMVLLVTAAN LGKMNRSCQS EKLIGYTKTV

301 TEVLAFLHCC LNPVLYAFIG QKFRNYFLKI LKDLWCVRRK YKSSGFSCAG RYSENISRQT

361 SETADNDNAS SFTM

48 CCR6b 369aa

1 MNFSDVFDSS EDYFVSVNTS YYSVDSEMLL CSLQEVRQFS RLFVPIAYSL ICVFGLLGNI

61 LVVITFAFYK KARSMTDVYL LNMAIADILF VLTLPFWAVS HATGAWVFSN ATCKLLKGIY

121 AINFNCGMLL LTCISMDRYI AIVQATKSFR LRSRTLPRSK IICLVVWGLS VIISSSTFVF

181 NQKYNTQGSD VCEPKYQTVS EPIRWKLLML GLELLFGFFI PLMFMIFCYT FIVKTLVQAQ

241 NSKRHKAIRV IIAVVLVFLA CQIPHNMVLL VTAANLGKMN RSCQSEKLIG YTKTVTEVLA

301 FLHCCLNPVL YAFIGQKFRN YFLKILKDLW CVRRKYKSSG FSCAGRYSEN ISRQTSETAD

361 NDNASSFTM

49 CCR7 ACCESSION P32248 378aa

1 MDLGKPMKSV LVVALLVIFQ VCLCQDEVTD DYIGDNTTVD YTLFESLCSK KDVRNFKAWF

61 LPIMYSIICF VGLLGNGLVV LTYIYFKRLK TMTDTYLLNL AVADILFLLT LPFWAYSAAK

121 SWVFGVHFCK LIFAIYKMSF FSGMLLLLCI SIDRYVAIVQ AVSAHRHRAR VLLISKLSCV

181 GIWILATVLS IPELLYSDLQ RSSSEQAMRC SLITEHVEAF ITIQVAQMVI GFLVPLLAMS

241 FCYLVIIRTL LQARNFERNK AIKVIIAVVV VFIVFQLPYN GVVLAQTVAN FNITSSTCEL

301 SKQLNIAYDV TYSLACVRCC VNPFLYAFIG VKFRNDLFKL FKDLGCLSQE QLRQWSSCRH

361 IRRSSMSVEA ETTTRFSP

50 CCR8 ACCESSION P51685 355aa

1 MDYTLDLSVT TVTDYYYPDI FSSPCDAELI QTNGKLLLAV FYCLLFVFSL LGNSLVILVL

61 VVCKKLRSIT DVYLLNLALS DLLFVFSFPF QTYYLLDQWV FGTVMCKVVS GFYYIGFYSS

121 MFFITLMSVD RYLAVVHAVY ALKVRTIRMG TTLCLAVWLT AIMATIPLLV FYQVASEDGV

181 LQCYSFYNQQ TLKWKIFTNF KMNILGLLIP FTIFMFCYIK ILHQLKRCQN HNKTKAIRLV

241 LIVVIASLLF WVPFNVVLFL TSLHSMHILD GCSISQQLTY ATHVREIISF THCCVNPVIY

301 AFVGEKFKKH LSEIFQKSCS QIFNYLGRQM PRESCEKSSS CQQHSSRSSS VDYIL

51 CCR9a ACCESSION XP₁₃003251 369aa

1 MTPTDFTSPI PNMADDYGSE STSSMEDYVN FNFTDFYCEK NNVRQFASHF LPPLYWLVFI

61 VGALGNSLVI LVYWYCTRVK TMTDMFLLNL AIADLLFLVT LPFWAIAAAD QWKFQTFMCK

121 VVNSMYKMNF YSCVLLIMCI SVDRYIAIAQ AMPAHTWREK RLLYSKMVCF TIWVLAAALC

181 IPEILYSQIK EESGIAICTM VYPSDESTKL KSAVLTLKVI LGFFLPFVVM ACCYTIIIHT

241 LIQAKKSSKH KALKVTITVL TVFVLSQFPY NCILLVQTID AYAMFISNCA VSTNIDICFQ

301 VTQTIAFFHS CLNPVLYVFV GERFRRDLVK TLKNLGCISQ AQWVSFTRRE GSLKLSSMLL

361 ETTSGALSL

52 CCR9b ACCESSION P51686 357aa

1 MADDYGSEST SSMEDYVNFN FTDFYCEKNN VRQFASHFLP PLYWLVFIVG ALGNSLVILV

61 YWYCTRVKTM TDMFLLNLAI ADLLFLVTLP FWAIAAADQW KFQTFMCKVV NSMYKMNFYS

121 CVLLIMCISV DRYIAIAQAM RAHTWREKRL LYSKMVCFTI WVLAAALCIP EILYSQIKEE

181 SGIAICTMVY PSDESTKLKS AVLTLKVILG FFLPFVVMAC CYTIIIHTLI QAKKSSKHKA

241 LKVTITVLTV FVLSQFPYNC ILLVQTIDAY AMFISNCAVS TNIDICFQVT QTIAFFHSCL

301 NPVLYVFVGE RFRRDLVKTL KNLGCISQAQ WVSFTRREGS LKLSSMLLET TSGALSL

53 CCR10 ACCESSION P46092 362aa

1 MGTEATEQVS WGHYSGDEED AYSAEPLPEL CYKADVQAFS RAFQPSVSLT VAALGLAGNG

61 LVLATHLAAR RAARSPTSAH LLQLALADLL LALTLPFAAA GALQGWSLGS ATCRTISGLY

121 SASFHAGFLF LACISADRYV AIARALPAGP RPSTPGRAHL VSVIVWLLSL LLALPALLFS

181 QDGQREGQRR CRLIFPEGLT QTVKGASAVA QVALGFALPL GVMVACYALL GRTLLAARGP

241 ERRRALRVVV ALVAAFVVLQ LPYSLALLLD TADLLAARER SCPASKRKDV ALLVTSGLAL

301 ARCGLNPVLY AFLGLRFRQD LRRLLRGGSS PSGPQPRRGC PRRPRLSSCS APTETHSLSW

361 DN

54 CCR11 ACCESSION AAF61299 350aa

1 MALEQNQSTD YYYEENEMNG TYDYSQYELI CIKEDVREFA KVFLPVFLTI VFVIGLAGNS

61 MVVAIYAYYK KQRTKTDVYI LNLAVADLLL LFTLPFWAVN AVHGWVLGKI MCKITSALYT

121 LNFVSGMQFL ACISIDRYVA VTKVPSQSGV GKPCWIICFC VWMAAILLSI PQLVFYTVND

181 NARCIPIFPR YLGTSMKALI QMLEICIGFV VPFLIMGVCY FITARTLMKM PNIKISRPLK

241 VLLTVVIVFI VTQLPYNIVK FCRAIDIIYS LITSCNMSKR MDIAIQVTES IALFHSCLNP

301 ILYVFMGASF KNYVMKVAKK YGSWRRQRQS VEEFPFDSEG PTEPTSTFSI

55 CXCR1 ACCESSION P25024 350aa

1 MSNITDPQMW DFDDLNFTGM PPADEDYSPC MLETETLNKY VVIIAYALVF LLSLLGNSLV

61 MLVILYSRVG RSVTDVYLLN LALADLLFAL TLPIWAASKV NGWIFGTFLC KVVSLLKEVN

121 FYSGILLLAC ISVDRYLAIV HATRTLTQKR HLVKFVCLGC WGLSMNLSLP FFLFRQAYHP

181 NNSSPVCYEV LGNDTAKWRM VLRILPHTFG FIVPLFVMLF CYGFTLRTLF KAHMGQKHRA

241 MRVIFAVVLI FLLCWLPYNL VLLADTLMRT QVIQETCERR NNIGRALDAT EILGFLHSCL

301 NPIIYAFIGQ NFRHGFLKIL AMHGLVSKEF LARHRVTSYT SSSVNVSSNL

56 CXCR2 ACCESSION P25025 360aa

1 MEDFNMESDS FEDFWKGEDL SNYSYSSTLP PFLLDAAPCE PESLEINKYF VVIIYALVFL

61 LSLLGNSLVM LVILYSRVGR SVTDVYLLNL ALADLLFALT LPIWAASKVN GWIFGTFLCK

121 VVSLLKEVNF YSGILLLACI SVDRYLAIVH ATRTLTQKRY LVKFICLSIW GLSLLLALPV

181 LLFRRTVYSS NVSPACYEDM GNNTANWRML LRILPQSFGF IVPLLIMLFC YGFTLRTLFK

241 AHMGQKHRAM RVIFAWLIF LLCWLPYNLV LLADTLMRTQ VIQETCERRN HIDRALDATE

301 ILGILHSCLN PLIYAFIGQK FRHGLLKILA IHGLISKDSL PKDSRPSFVG SSSGHTSTTL

57 CXCR3 ACCESSION P49682 368aa

1 MVLEVSDHQV LNDAEVAALL ENFSSSYDYG ENESDSCCTS PPCPQDFSLN FDRAFLPALY

61 SLLFLLGLLG NGAVAAVLLS RRTALSSTDT FLLHLAVADT LLVLTLPLWA VDAAVQWVFG

121 SGLCKVAGAL FNINFYAGAL LLACISFDRY LNIVHATQLY RRGPPARVTL TCLAVWGLCL

181 LFALPDFIFL SAHHDERLNA THCQYNFPQV GRTALRVLQL VAGFLLPLLV MAYCYAHILA

241 VLLVSRGQRR LRAMRLVVVV VVAFALCWTP YHLVVLVDIL MDLGALARNC GRESRVDVAK

301 SVTSGLGYMH CCLNPLLYAF VGVKFRERMW MLLLRLGCPN QRGLQRQPSS SRRDSSWSET

361 SEASYSGL

58 CXCR4 ACCESSION P30991 352aa

1 MEGISIYTSD NYTEEMGSGD YDSMKEPCFR EENANFNKIF LPTIYSIIFL TGIVGNGLVI

61 LVMGYQKKLR SMTDKYRLHL SVADLLFVIT LPFWAVDAVA NWYFGNFLCK AVHVIYTVNL

121 YSSVLILAFI SLDRYLAIVH ATNSQRPRKL LAEKVVYVGV WIPALLLTIP DFIFANVSEA

181 DDRYICDRFY PNDLWVVVFQ FQHIMVGLIL PGIVILSCYC IIISKLSHSK GHQKRKALKT

241 TVILILAFFA CWLPYYIGIS IDSFILLEII KQGCEFENTV HKWISITEAL AFFHCCLNPI

301 LYAFLGAKFK TSAQHALTSV SRGSSLKILS KGKRGGHSSV STESESSSFH SS

59 CXCR5 ACCESSION P32302 372aa

1 MNYPLTLEMD LENLEDLFWE LDRLDNYNDT SLVENHLCPA TEGPLMASFK AVFVPVAYSL

61 IFLLGVIGNV LVLVILERHR QTRSSTETFL FHLAVADLLL VFILPFAVAE GSVGWVLGTF

121 LCKTVIALHK VNFYCSSLLL ACIAVDRYLA IVHAVHAYRH RRLLSIHITC GTIWLVGFLL

181 ALPEILFAKV SQGHHNNSLP RCTFSQENQA ETHAWFTSRF LYHVAGFLLP MLVMGWCYVG

241 VVHRLRQAQR RPQRQKAVRV AILVTSIFFL CWSPYHIVIF LDTLARLKAV DNTCKLNGSL

301 PVAITMCEFL GLAHCCLNPM LYTFAGVKFR SDLSRLLTKL GCTGPASLCQ LFPSWRRSSL

361 SESENATSLT TF

60 CXCR6 ACCESSION NP₁₃006555 342aa

1 MAEHDYHEDY GFSSFNDSSQ EEHQDFLQFS KVFLPCMYLV VFVCGLVGNS LVLVISIFYH

61 KLQSLTDVFL VNLPLADLVF VCTLPFWAYA GIHEWVFGQV MCKSLLGIYT INFYTSMLIL

121 TCITVDRFIV VVKATKAYNQ QAKRMTWGKV TSLLIWVISL LVSLPQIIYG NVFNLDKLIC

181 GYHDEAISTV VLATQMTLGF FLPLLTMIVC YSVIIKTLLH AGGFQKHRSL KIIFLVMAVF

241 LLTQMPFNLM KFIRSTHWEY YAMTSFHYTI MVTEAIAYLR ACLNPVLYAF VSLKFRKNFW

301 KLVKDIGCLP YLGVSHQWKS SEDNSKTFSA SHNVEATSMF QL

61 CX3CR1 ACCESSION P49238 355aa

1 MDQFPESVTE NFEYDDLAEA CYIGDIVVFG TVFLSIFYSV IFAIGLVGNL LVVFALTNSK

61 KPKSVTDIYL LNLALSDLLF VATLPFWTHY LINEKGLHNA MCKFTTAFFF IGFFGSIFFI

121 TVISIDRYLA IVLAANSMNN RTVQHGVTIS LGVWAAAILV AAPQFMFTKQ KENECLGDYP

181 EVLQEIWPVL RNVETNFLGF LLPLLIMSYC YFRIIQTLFS CKNHKKAKAI KLILLVVIVF

241 FLFWTPYNVM IFLETLKLYD FFPSCDMRKD LRLALSVTET VAFSHCCLNP LIYAFAGEKF

301 RRYLYHLYGK CLAVLCGRSV HVDFSSSESQ RSRHGSVLSS NFTYHTSDGD ALLLL

62 XCR1 ACCESSION P46094 333aa

1 MESSGNPEST TFFYYDLQSQ PCENQAWVFA TLATTVLYCL VFLLSLVGNS LVLWVLVKYE

61 SLESLTNIFI LNLCLSDLVF ACLLPVWISP YHWGWVLGDF LCKLLNMIFS ISLYSSIFFL

121 TIMTIHRYLS VVSPLSTLRV PTLRCRVLVT MAVWVASILS SILDTIFHKV LSSGCDYSEL

181 TWYLTSVYQH NLFFLLSLGI ILFCYVEILR TLFRSRSKRR HRTVKLIFAI VVAYFLSWGP

241 YNFTLFLQTL FRTQIIRSCE AKQQLEYALL ICRNLAFSHC CFNPVLYVFV GVKFRTHLKH

301 VLRQFWFCRL QAPSPASIPH SPGAFAYEGA SFY

63 D6 ACCESSION XP₁₃003126 384aa

1 MAATASPQPL ATEDADSENS SFYYYDYLDE VAFMLCRKDA VVSFGKVFLP VFYSLIFVLG

61 LSGNLLLLMV LLRYVPRRRM VEIYLLNLAI SNLLFLVTLP FWGISVAWHW VFGSFLCKMV

121 STLYTINFYS GIFFISCMSL DKYLEIVHAQ PYHRLRTRAK SLLLATIVWA VSLAVSIPDM

181 VFVQTHENPK GVWNCHADFG GHGTIWKLFL RFQQNLLGFL LPLLAMIFFY SRIGCVLVRL

241 RPAGQGRALK IAAALVVAFF VLWFPYNLTL FLHTLLDLQV FGNCEVSQHL DYALQVTESI

301 AFLHCCFSPI LYAFSSHRFR QYLKAFLAAV LGWHLAPGTA QASLSSCSES SILTAQEEMT

361 GMNDLGERQS ENYPNKEDVG NKSA

The medicaments according to the invention can be administered in suitable galenic dosage forms, especially in a lyophilized form taken up with mannitol or similar sugars in sterile ampoules for dissolving in physiological saline and/or infusion solution for repeated single injection and/or permanent infusion in amounts of from 300 mg to 30 mg of pure chemokine receptor ligands, especially chemokines, chemokine agonists or antagonists as well as chemokine and chemokine receptor antibodies, per therapeutic unit. Preferably, the medicament according to the invention is administered in a galenic dosage form in which the medicament is employed in biocompatible microspheres, systemically or topically through an aerosol or through intravenous or subcutaneous administration.

According to the invention, the following approach may also be used in this method. The tumor cells whose receptor composition is to be examined are grown in parallel in vitro, and the cells obtained therefrom are also examined for their receptor composition and treated with chemokines, preferably of the types HCC-1, HCC-2, MCP-1, RANTES or SDF-1. In addition, the known analogues were also employed. With the modified Boyden migration chamber method, it can be established that the tumor cells display a chemotactic response upon the addition of agonists which bind to the corresponding chemokine receptors. The inhibition of their migration is confirmed by previous incubation with antagonists or receptor antibodies.

When highly purified antibodies were used, it could be established that these are capable of causing apoptosis of tumor cells in both in-vitro and in-vivo models. When cell lines or removed tumor cells are grown using the usual cell culture methods, their survival time is highly reduced in vitro by the addition of chemokine antibodies whose corresponding receptors were previously detected on the cell surface of these cell types. The apoptosis of a large number of these cultivated cells can be observed.

Also with in-vivo models, surprisingly, a decrease in tumor cells by apoptosis can be observed.

Since nude mice have a deficient immune system, the metastatic spread behavior in a host body can be examined in a nude mouse model without the occurrence of the immune reactions which are known between species and without rejection of the foreign cells. Nude mice are inoculated in a per se known manner with tumor cells or tumor cell lines whose chemokine receptor distribution pattern had been analyzed, and the metastatic spread of these cells was checked upon treatment with chemokines and upon treatment with chemokine antagonists and/or receptor antibodies.

Surprisingly, it is found that chemokine antagonists and receptor antibodies belonging to the receptors found significantly inhibit or prevent metastatic spread while the addition of chemokines results in a modulation of tumor growth. Surprisingly, it is also found that the preparations analyzed by immunohistochemistry exhibit a specific distribution of chemokines and chemokine receptors in the tumor and the tumor-surrounding tissue. Thus, further selective targets have been recognized.

This intervention in accordance with the method according to the invention consists in additionally extending the proteome analysis of the chemokines and their receptors by the analysis of antitumoral peptides/proteins in the same method. This results in an extension of the diagnostic and therapeutic approach, especially to employ antagonists directed against further clusters of the tumor cell surface proteome. An enhancement of these effects can be achieved by combination with chemokine receptor antagonists and antibodies.

To confirm these results, tumor cell lines (e.g., preferably, LNCaP-, PC-3-, DU-145, HT-29-, Caco-2-, T-84-) may also be stably transfected with one or more of the chemokine receptors. Upon subsequent injection of these cells into animals in which a chemokine corresponding to the receptor is transgenically overexpressed in the liver, such tumor cells will settle. Thus, such modified cells preferably form metastases in the liver.

When the method is used in samples of effector cells in inflammatory (acute rejection in kidney transplantation) and auto-immune diseases (rheumatoid arthritis), a specific composition of the chemokine/chemokine receptor proteome, especially the chemokine receptors CXCR4 and CCR5 and/or CXCR3, can also be detected in the rejection of kidney grafts. It has also been demonstrated by in-vitro experiments that a mixture of antibodies and antagonists directed against these receptors strongly inhibits the migration of disease-specific effector cells.

In the following, the invention is illustrated in more detail by way of Examples.

### EXAMPLE 1

Preparation of specific antibodies against chemokine receptors (preferably CCR and CXCR) and other clusters of the tumor cell surface proteome.

Surprisingly, for the preparation of specific antibodies against chemokine receptors, it was found that the use of specific amino acid sequences (see Tables 1 and 2), especially against (1) the N-terminal extracellular and (2) the second extracellular loop domains of these 7-TMD receptors (seven transmembrane domains), is especially useful for immunization if the synthesized peptides are coupled to carrier molecules by the usual methods and injected into mice. In addition, multiple antigenic peptides (MAP) connected through lysine to form larger molecules or cell lines transfected with chemokine receptor (sequences according to table 3) are also suitable for preparing these antibodies. The use of immunogens from stably transfected cells bearing chemokin receptors has also proven surprisingly useful as a further method, wherein membrane isolates, cell extracts with complete or fragmented receptors or even lyophilized whole preparations were used.

The mice (type NZW×NZB) were employed for the preparation of monoclonal antibodies, which is effected with the routine methods of the IPF PharmaCeuticals GmbH. The antibodies checked with Western blot and ELISA can be employed for the diagnostic and therapeutic purposes mentioned when they have been highly purified with the laboratory methods of the IPF PharmaCeuticals GmbH. In detail, the sequences employed for the chemokine receptors are to be selected in accordance with sequences ID 1-63.

### EXAMPLE 2

Detection of chemokines and chemokine receptors in primary tumors, tumor metastases and single tumor cells.

In the surgical treatment of tumors (e.g., colon, prostate) and their metastases which were removed, for example, from the liver or lymph nodes, the tumor cells could be recovered and analyzed by immunohistochemical methods as well as further molecular-biological methods. In the immunochemical and molecular-biological analyses, it was established that the primary tumor cells, the metastases and circulating single cells (obtained from the blood of patients) contain a specific composition of chemokines and chemokine receptors. The algorithm of this composition is of high specificity individually and depending on the tumor, which surprisingly enables a selective tumor treatment on the basis of the diagnosed proteome clusters by the method according to the invention. Surprisingly, the algorithms of the chemokine receptors are preferably suitable. These algorithms derived from the experiments are defined as follows according to the invention:

X=chemokine receptors to be newly identified

n=0 to ∞ further chemokine receptors or chemokine receptors to be newly identified

| | | |
|---|---|---|
| CCR1 + CCR2 + n | CCR2 + CCR5 + n | CCR3 + CCR8 + n |
| CCR1 + CCR3 + n | CCR2 + CCR6 + n | CCR3 + CCR9 + n |
| CCR1 + CCR4 + n | CCR2 + CCR7 + n | CCR3 + CCR10 + n |
| CCR1 + CCR5 + n | CCR2 + CCR8 + n | CCR3 + CCR11 + n |
| CCR1 + CCR6 + n | CCR2 + CCR9 + n | CCR3 + CXCR1 + n |
| CCR1 + CCR7 + n | CCR2 + CCR10 + n | CCR3 + CXCR2 + n |
| CCR1 + CCR8 + n | CCR2 + CCR11 + n | CCR3 + CXCR3 + n |
| CCR1 + CCR9 + n | CCR2 + CXCR1 + n | CCR3 + CXCR4 + n |
| CCR1 + CCR10 + n | CCR2 + CXCR2 + n | CCR3 + CXCR5 + n |
| CCR1 + CCR11 + n | CCR2 + CXCR3 + n | CCR3 + CXCR6 + n |
| CCR1 + CXCR1 + n | CCR2 + CXCR4 + n | CCR3 + XCR1 + n |
| CCR1 + CXCR2 + n | CCR2 + CXCR5 + n | CCR3 + CX3CR1 + n |
| CCR1 + CXCR3 + n | CCR2 + CXCR6 + n | CCR3 + D6 + n |
| CCR1 + CXCR4 + n | CCR2 + XCR1 + n | CCR3 + X + n |
| CCR1 + CXCR5 + n | CCR2 + CX3CR1 + n | |
| CCR1 + CXCR6 + n | CCR2 + D6 + n | CCR4 + CCR1 + n |
| CCR1 + XCR1 + n | CCR2 + X + n | CCR4 + CCR2 + n |
| CCR1 + CX3CR1 + n | | CCR4 + CCR3 + n |
| CCR1 + D6 + n | CCR3 + CCR1 + n | CCR4 + CCR5 + n |
| CCR1 + X + n | CCR3 + CCR2 + n | CCR4 + CCR6 + n |
| | CCR3 + CCR4 + n | CCR4 + CCR7 + n |
| CCR2 + CCR1 + n | CCR3 + CCR5 + n | CCR4 + CCR8 + n |
| CCR2 + CCR3 + n | CCR3 + CCR6 + n | CCR4 + CCR9 + n |
| CCR2 + CCR4 + n | CCR3 + CCR7 + n | CCR4 + CCR10 + n |
| CCR4 + CCR11 + n | CCR6 + CCR9 + n | CCR8 + CCR6 + n |
| CCR4 + CXCR1 + n | CCR6 + CCR10 + n | CCR8 + CCR7 + n |
| CCR4 + CXCR2 + n | CCR6 + CCR11 + n | CCR8 + CCR9 + n |
| CCR4 + CXCR3 + n | CCR6 + CXCR1 + n | CCR8 + CCR10 + n |
| CCR4 + CXCR4 + n | CCR6 + CXCR2 + n | CCR8 + CCR11 + n |
| CCR4 + CXCR5 + n | CCR6 + CXCR3 + n | CCR8 + CXCR1 + n |
| CCR4 + CXCR6 + n | CCR6 + CXCR4 + n | CCR8 + CXCR2 + n |
| CCR4 + XCR1 + n | CCR6 + CXCR5 + n | CCR8 + CXCR3 + n |
| CCR4 + CX3CR1 + n | CCR6 + CXCR6 + n | CCR8 + CXCR4 + n |
| CCR4 + D6 + n | CCR6 + XCR1 + n | CCR8 + CXCR5 + n |
| CCR4 + X + n | CCR6 + CX3CR1 + n | CCR8 + CXCR6 + n |
| | CCR6 + D6 + n | CCR8 + XCR1 + n |
| CCR5 + CCR1 + n | CCR6 + X + n | CCR8 + CX3CR1 + n |
| CCR5 + CCR2 + n | | CCR8 + D6 + n |
| CCR5 + CCR3 + n | CCR7 + CCR1 + n | CCR8 + X + n |
| CCR5 + CCR4 + n | CCR7 + CCR2 + n | |
| CCR5 + CCR6 + n | CCR7 + CCR3 + n | CCR9 + CCR1 + n |
| CCR5 + CCR7 + n | CCR7 + CCR4 + n | CCR9 + CCR2 + n |
| CCR5 + CCR8 + n | CCR7 + CCR5 + n | CCR9 + CCR3 + n |
| CCR5 + CCR9 + n | CCR7 + CCR6 + n | CCR9 + CCR4 + n |
| CCR5 + CCR10 + n | CCR7 + CCR8 + n | CCR9 + CCRS + n |
| CCR5 + CCR11 + n | CCR7 + CCR9 + n | CCR9 + CCR6 + n |
| CCR5 + CXCR1 + n | CCR7 + CCR10 + n | CCR9 + CCR7 + n |
| CCR5 + CXCR2 + n | CCR7 + CCR11 + n | CCR9 + CCR8 + n |
| CCR5 + CXCR3 + n | CCR7 + CXCR1 + n | CCR9 + CCR10 + n |
| CCR5 + CXCR4 + n | CCR7 + CXCR2 + n | CCR9 + CCR11 + n |
| CCR5 + CXCR5 + n | CCR7 + CXCR3 + n | CCR9 + CXCR1 + n |
| CCR5 + CXCR6 + n | CCR7 + CXCR4 + n | CCR9 + CXCR2 + n |
| CCR5 + XCR1 + n | CCR7 + CXCR5 + n | CCR9 + CXCR3 + n |
| CCR5 + CX3CR1 + n | CCR7 + CXCR6 + n | CCR9 + CXCR4 + n |
| CCR5 + D6 + n | CCR7 + XCR1 + n | CCR9 + CXCR5 + n |
| CCR5 + X + n | CCR7 + CX3CR1 + n | CCR9 + CXCR6 + n |
| | CCR7 + D6 + n | CCR9 + XCR1 + n |
| CCR6 + CCR1 + n | CCR7 + X + n | CCR9 + CX3CR1 + n |
| CCR6 + CCR2 + n | | CCR9 + D6 + n |
| CCR6 + CCR3 + n | CCR8 + CCR1 + n | CGR9 + X + n |
| CCR6 + CCR4 + n | CCR8 + CCR2 + n | |
| CCR6 + CCR5 + n | CCR8 + CCR3 + n | CCR10 + CCR1 + n |
| CCR6 + CCR7 + n | CCR8 + CCR4 + n | CCR10 + CCR2 + n |
| CCR6 + CCR8 + n | CCR8 + CCR5 + n | CCR10 + CCR3 + n |
| CCR10 + CCR4 + n | CXCR1 + CCR2 + n | |
| CCR10 + CCR5 + n | CXCR1 + CCR3 + n | CXCR3 + CCR1 + n |
| CCR10 + CCR6 + n | CXCR1 + CCR4 + n | CXCR3 + CCR2 + n |
| CCR10 + CCR7 + n | CXCR1 + CCR5 + n | CXCR3 + CCR3 + n |
| CCR10 + CCR8 + n | CXCR1 + CCR6 + n | CXCR3 + CCR4 + n |
| CCR10 + CCR9 + n | CXCR1 + CCR7 + n | CXCR3 + CCR5 + n |
| CCR10 + CCR11 + n | CXCR1 + CCR8 + n | CXCR3 + CCR6 + n |
| CCR10 + CXCR1 + n | CXCR1 + CCR9 + n | CXCR3 + CCR7 + n |
| CCR10 + CXCR2 + n | CXCR1 + CCR10 + n | CXCR3 + CCR8 + n |
| CCR10 + CXCR3 + n | CXCR1 + CCR11 + n | CXCR3 + CCR9 + n |
| CCR10 + CXCR4 + n | CXCR1 + CXCR2 + n | CXCR3 + CCR10 + n |
| CCR10 + CXCR5 + n | CXCR1 + CXCR3 + n | CXGR3 + CCR11 + n |
| CCR10 + CXCR6 + n | CXCR1 + CXCR4 + n | CXCR3 + CXCR1 + n |
| CCR10 + XCR1 + n | CXCR1 + CXCR5 + n | CXCR3 + CXCR2 + n |
| CCR10 + CX3CR1 + | CXCR1 + CXCR6 + n | CXCR3 + CXCR4 + n |
| n | | |
| CCR10 + D6 + n | CXCR1 + XCR1 + n | CXCR3 + CXCR5 + n |
| CCR10 + X + n | CXCR1 + CX3CR1 + | CXCR3 + CXCR6 + n |
| | n | |
| | CXCR1 + D6 + n | CXCR3 + XCR1 + n |
| CCR11 + CCR1 + n | CXCR1 + X + n | CXCR3 + CX3CR1 + n |
| CCR11 + CCR3 + n | | CXCR3 + D6 + n |
| CCR11 + CCR4 + n | CXCR2 + CCR1 + n | CXCR3 + X + n |
| CCR11 + CCR5 + n | CXCR2 + CCR2 + n | |
| CCR11 + CCR6 + n | CXCR2 + CCR3 + n | CXCR4 + CCR1 + n |
| CCR11 + CCR7 + n | CXCR2 + CCR4 + n | CXCR4 + CCR2 + n |
| CCR11 + CCR8 + n | CXCR2 + CCR5 + n | CXCR4 + CCR3 + n |
| CCR11 + CCR9 + n | CXCR2 + CCR6 + n | CXCR4 + CCR4 + n |
| CCR11 + CCR10 + n | CXCR2 + CCR7 + n | CXCR4 + CCR5 + n |
| CCR11 + CCR11 + n | CXCR2 + CCR8 + n | CXCR4 + CCR6 + n |
| CCR11 + CXCR1 + n | CXCR2 + CCR9 + n | CXCR4 + CCR7 + n |
| CCR11 + CXCR2 + n | CXCR2 + CCR10 + n | CXCR4 + CCR8 + n |
| CCR11 + CXCR3 + n | CXCR2 + CCR11 + n | CXCR4 + CCR9 + n |
| CCR11 + CXCR4 + n | CXCR2 + CXCR1 + n | CXCR4 + CCR10 + n |
| CCR11 + CXCR5 + n | CXCR2 + CXCR3 + n | CXCR4 + CCR11 + n |
| CCR11 + CXCRG + n | CXCR2 + CXCR4 + n | CXCR4 + CXCR1 + n |
| CCR11 + XCR1 + n | CXCR2 + CXCR5 + n | CXCR4 + CXCR2 + n |
| CCR11 + CX3CR1 + | CXCR2 + CXCR6 + n | CXCR4 + CXCR3 + n |
| n | | |
| CCR11 + D6 + n | CXCR2 + XCR1 + n | CXCR4 + CXCR5 + n |
| CCR11 + X + n | CXCR2 + CX3CR1 + | CXCR4 + CXCR6 + n |
| | n | |
| | CXCR2 + D6 + n | CXCR4 + XCR1 + n |
| CXCR1 + CCR1 + n | CXCR2 + X + n | CXCR4 + CX3CR1 + n |
| CXCR4 + D6 + n | CXCR6 + XCR1 + n | CX3CR1 + CXCR4 + n |
| CXCR4 + X + n | CXCR6 + CX3CR1 + | CX3CR1 + CXCR5 + n |
| | n | |
| | CXCR6 + D6 + n | CX3CR1 + CXCR6 + n |
| CXCR5 + CCR1 + n | CXCR6 + X + n | CX3CR1 + XCR1 + n |
| CXCR5 + CCR2 + n | | CX3CR1 + D6 + n |
| CXCR5 + CCR3 + n | XCR1 + CCR1 + n | CX3CR1 + X + n. |
| CXCR5 + CCR4 + n | XCR1 + CCR2 + n | |
| CXCR5 + CCR5 + n | XCR1 + CCR3 + n | |
| CXCR5 + CCR6 + n | XCR1 + CCR4 + n | |
| CXCR5 + CCR7 + n | XCR1 + CCR5 + n | |
| CXCR5 + CCR8 + n | XCR1 + CCR6 + n | |
| CXCR5 + CCR9 + n | XCR1 + CCR7 + n | |
| CXCR5 + CCR10 + n | XCR1 + CCR8 + n | |
| CXCR5 + CCR11 + n | XCR1 + CCR9 + n | |
| CXCR5 + CXCR1 + n | XCR1 + CCR10 + n | |
| CXCR5 + CXCR2 + n | XCR1 + CCR11 + n | |
| CXCR5 + CXCRJ + n | XCR1 + CXCR1 + n | |
| CXCR5 + CXCR4 + n | XCR1 + CXCR2 + n | |
| CXCR5 + CXCR6 + n | XCR1 + CXCR3 + n | |
| CXCR5 + XCR1 + n | XCR1 + CXCR4 + n | |
| CXCR5 + CX3CR1 + | XCR1 + CXCR5 + n | |
| n | | |
| CXCR5 + D6 + n | XCR1 + CXCR6 + n | |
| CXCR5 + X + n | XCR1 + CX3CR1 + n | |
| | XCR1 + D6 + n | |
| CXCR6 + CCR1 + n | XCR1 + X + n | |
| CXCR6 + CCR2 + n | | |
| CXCR6 + CCR3 + n | CX3CR1 + CCR1 + n | |
| CXCR6 + CCR4 + n | CX3CR1 + CCR2 + n | |
| CXCR6 + CCRS + n | CX3CR1 + CCR3 + n | |
| CXCR6 + CCR6 + n | CX3CR1 + CCR4 + n | |
| CXCR6 + CCR7 + n | CX3CR1 + CCR5 + n | |
| CXCR6 + CCR8 + n | CX3CR1 + CCR6 + n | |
| CXCR6 + CCR9 + n | CX3CR1 + CCR7 + n | |
| CXCR6 + CCR10 + n | CX3CR1 + CCR8 + n | |
| CXCR6 + CCR11 + n | CX3CR1 + CCR9 + n | |
| CXCR6 + CXCR1 + n | CX3CR1 + CCR10 + | |
| | n | |
| CXCR6 + CXCR2 + n | CX3CR1 + CCR11 + | |
| | n | |
| CXCR6 + CXCR3 + n | CX3CR1 + CXCR1 + | |
| | n | |
| CXCR6 + CXCR4 + n | CX3CR1 + CXCR2 + | |
| | n | |
| CXCR6 + CXCR5 + n | CX3CR1 + CXCR3 + | |
| | n | |

1. A diagnostic agent containing at least two different ligands of receptors involved in a pathological process.

2. A diagnostic agent containing at least two different chemokine receptor ligands.

3. The diagnostic agent according to claim 2, wherein said chemokine receptor ligands are chemokines, chemokine derivatives, agonists or antagonists of chemokine receptors, antibodies or antibody fragments which at least partially block the binding site of the chemokine receptor.

4. The diagnostic agent according to claims 2 and 3, wherein said chemokines are selected from the group consisting of C, CC, CXC, CX₃C chemokines, their pharmacological analogues, binding proteins and antibodies which bind to the specific receptors in accordance with the chemokines mentioned.

5. Use of at least two different ligands of receptors involved in a pathological process for the diagnosis of diseases.

6. The use according to claim 5, wherein at least two chemokine receptor ligands and/or two different chemokine receptors are employed for the diagnosis of tumors.

7. The use according to claim 6, wherein said tumors are selected from the group consisting of colorectal tumors and prostatic tumors.

8. Use of at least two different chemokine receptor ligands and/or two different chemokine receptors for the diagnosis of organ rejection reactions.

9. Use of at least two different chemokine receptor ligands and/or two different chemokine receptors for the diagnosis of inflammatory processes.

10. Use of at least two different chemokine receptor ligands and/or two different chemokine receptors for the diagnosis of auto-immune diseases.

11. A medicament containing at least one inhibitor of at least two different ligands of receptors involved in a pathological process.

12. The medicament according to claim 11, containing at least one inhibitor of at least two chemokine receptors.

13. The medicament according to claim 12, containing antagonists of chemokine receptors, antibodies or antibody fragments which at least partially block the binding site of the chemokine receptor.

14. Use of inhibitors of at least two different ligands of receptors involved in a pathological process for the treatment of such diseases.

15. The use according to claim 14, wherein at least two chemokine receptors are used for preparing a medicament for the treatment of tumors, inflammatory processes, auto-immune diseases, diseases of the bone marrow.

16. The use according to claim 14, wherein tumors, inflammatory processes, auto-immune diseases of the vascular system, lymph system, respiratory tract, digestive tract and urogenital tract including the kidney are involved.

17. The use according to claims 15 and 16, wherein said inhibitors are selected from the group antagonists of chemokine receptors, antibodies or antibody fragments which at least partially block the binding sites of the chemokine receptor and thus modulate their function.

18. The use according to any of claims 15 to 17, wherein said tumors are selected from the group consisting of colorectal tumors, prostatic tumors and other tumor diseases of the blood system, lymph system, cardiovascular system, nervous system, respiratory tract, digestive tract, endocrine system, skin including integumentary appendages, locomotor system and urogenital tract including the kidney.

19. The use according to any of claims 15 to 17, wherein said inflammatory processes are selected from the group consisting of asthma bronchiale, chronic inflammatory bowel diseases, organ rejection and further inflammatory processes of the blood system, lymph system, cardiovascular system, nervous system, respiratory tract, digestive tract, endocrine system, skin including integumentary appendages, locomotor system and urogenital tract including the kidney.

20. The use according to any of claims 15 to 17, wherein said auto-immune diseases are selected from the group consisting of rheumatoid arthritis, lupus erythematodes and other chronic diseases of the blood system, lymph system, cardiovascular system, nervous system, respiratory tract, digestive tract, endocrine system, skin including integumentary appendages, locomotor system and urogenital tract including the kidney.

21. Use of at least one chemokine receptor ligand and/or chemokine receptor for the diagnosis of organ rejection reactions following organ transplantations.

22. Use of an inhibitor of a chemokine receptor for preparing a medicament for preventing or alleviating organ rejection reactions following organ transplantations.

23. The use according to claim 22, following transplantations of the liver, kidney, pancreas, small intestine, as well as other organs, tissues and cell systems of the gastrointestinal tract, respiratory tract, urogenital tract, cardiovascular system, neuro-endocrine system, and the locomotor system as well as the blood and immune systems.

24. Peptides having the SEQ ID NOS. 1 to 40.

25. Use of the peptides according to claim 24 for preparing antibodies against a chemokine receptor.

26. A method for recognizing receptors involved in pathological processes, wherein expression profiles are examined on the proteome level using cell-biological or cytochemical methods, especially by immunochemical methods, immunohistochemistry using serial sections or multiple successive or simultaneous single sections, FACS analysis and/or the expression of receptors on the transcriptional level by molecular-biological methods, especially PCR, Northern analysis and/or in-situ hybridization methods.

27. The method according to claim 26, wherein a diagnostic agent according to any of claims 1 to 4 is employed.
